(19) United States
(12) Patent Application Publication
(10) Pub. No.: US 2022/0105290 A1
(43) Pub. Date: Apr. 7, 2022

(54) APPARATUS AND INSTALLATION FOR SUPPLYING THERAPEUTIC GAS TO A PATIENT WITH FLOW CONTROL

(71) Applicant: L'Air Liquide, Société Anonyme pour l'Etude et l'Exploitation des Procédés Georges Claude, Paris (FR)

(72) Inventor: Thierry BOULANGER, Philadelphia, PA (US)

(21) Appl. No.: 17/494,540

(22) Filed: Oct. 5, 2021

(30) Foreign Application Priority Data
Oct. 6, 2020 (FR) 2010172

(51) Int. Cl. A61M 16/00; A61M 16/20; A61M 16/06

(52) U.S. Cl. CPC A61M 16/024; A61M 16/0003; A61M 2016/003; A61M 16/06; A61M 2016/0027; A61M 16/208

(57) ABSTRACT

The invention relates to a gas delivery apparatus comprising an internal gas passage, a deformable reservoir, a valve device arranged upstream of the deformable reservoir and a control unit with microprocessor controlling the valve device in order to set or adjust the gas flow. A pressure sensor performs gas pressure measurements in a respiratory mask and supplies them to the control unit. The control unit compares the pressure measurements to a pressure threshold value and controls the valve device in order to adjust the gas flow as a function of this comparison, particularly in order to increase the flow when the pressure measured is below the threshold value.

### CROSS REFERENCE TO RELATED APPLICATIONS

This application claims the benefit of priority under 35 U.S.C. § 119 (a) and (b) to French Patent Application No. 2010172, filed Oct. 6, 2020, the entire contents of which are incorporated herein by reference.

### BACKGROUND

The invention relates to an apparatus for delivering gas and an installation for supplying therapeutic gas incorporating such a gas delivery apparatus that can be used to supply a therapeutic gas (i.e. pure gas or a gas mixture) to a conscious patient in different care premises, in particular in hospital, including for inhalation for a long period, for example several hours, while minimizing gas losses.

Certain therapies require that therapeutic gas made up of a mixture of several gaseous ingredients be administered to conscious patients.

It is thus known practice to use an equimolar mixture (50%/50%) of nitrous oxide (N₂O) and oxygen (O₂) to reduce anxiety, produce a sedative effect and/or attenuate acute pain.

Likewise, the use of a mixture of argon and oxygen (60 vol % Ar/40 vol % O₂) has been proposed, inhaled before and during, or even after, a mechanical thrombectomy procedure, in order to treat ischaemic strokes.

Although the administration of the gas by inhalation is generally short, that is, typically less than 30 minutes, longer inhalations, of the order of 1 hour or more, are sometimes necessary.

Generally, the therapeutic gas (i.e. one or more ingredients) is inhaled via a respiratory mask, typically a face mask, i.e. naso-buccal, by a conscious patient, either continuously or intermittently, that is, periodically.

During continuous administration, a continuous flow of gas, exceeding the minute ventilation of the patient (i.e. the mean volume of gas inhaled by the patient in one minute) is delivered continuously during the inspiratory and expiratory phases of the patient. During the inspiratory phases, the patient inhales the gas contained in a deformable reservoir, while during the expiratory phases, the gas fills the deformable reservoir again in order to prepare for the next inspiratory phase.

However, continuous administration has drawbacks, in particular:
the minute ventilation of the patient must be checked regularly in order to adjust the gas flow, if necessary. This requires the almost permanent presence of a health worker, which is not possible for patients being treated at home and is complicated in a hospital environment, where workers must look after several patients at once.
the flow selected must be greater than the minute ventilation of the patient in order to prevent the reservoir from emptying over time. As a result, the flow is always set higher than necessary. This leads to significant losses of gas, which escapes into the atmosphere, without having been inhaled by the patient.
in the case of prolonged inhalation, for example during surgery lasting more than two hours, in order to handle any variation in the minute ventilation of the patient, the continuous gas flow is set to a conservative value, which is generally far greater than the mean minute ventilation of the patient. The volumes of gas necessary are therefore very significant, which results in complicated logistics and problems of space in the operating room or similar: frequent replacements of empty gas cylinders with full ones, storage of empty and full cylinders, lack of free space for storage, etc.

The intermittent, that is, non-continuous delivery of gas, is therefore often preferred. To do this, devices known as “demand valves” are conventionally used.

During the inspiratory phases, a demand valve or DV opens and delivers therapeutic gas in proportion to the negative pressure generated by the patient's inspirations in the respiratory mask with which the patient is provided, while during the expiratory phases, the DV closes and stops delivering gas.

A DV makes it possible to supply only the quantity of gas that the patient needs, that is, exactly their minute ventilation. This makes it possible to greatly limit gas consumption and therefore avoid the aforementioned drawbacks that exist during continuous administration.

However, a DV also has drawbacks. It thus requires a relatively significant minimum negative pressure in order to open and supply the gas flow to the patient and, once open, a considerable inspiratory effort on the part of the patient is necessary in order to inspire the gas they need.

For these reasons, DVs are contra-indicated for certain patients, in particular frail people (e.g. infants, the elderly, etc.), and are not therefore currently suitable for treating certain pathologies affecting these types of patient, for example those suffering from a stroke, the majority of whom are elderly.

In addition, another problem exists with both systems (i.e. continuous and discontinuous) in the treatment of certain pathologies. Due to space constraints relating to the act of mechanical thrombectomy in the treatment of strokes, the administration system must thus be placed a long distance away from the patient's head and connected to the patient by means of gas supply pipes, which can be up to several metres in length, typically up to four metres, or even more.

These pipes generate additional resistance to inspiration, that is, the patient must produce even greater negative pressure in order to be able to inspire gas and meet their ventilation needs.

If there are “leaks” on the mask, i.e. defective sealing on the perimeter of the mask in contact with the face, this negative pressure draws in ambient air and dilutes the inhaled gas.

This undesired dilution of the therapeutic gas with ambient air is very problematic, as it can lead to a significant reduction in efficacy of the medical gas, i.e. of the therapeutic gas, administered to the patient.

This is medically unacceptable and it is therefore necessary to minimize this negative pressure as far as possible in order to limit the dilution of the therapeutic gas with ambient air, at the risk of not obtaining the expected clinical benefit, that is, of not treating the patient effectively.

In this context, one problem is that of proposing an apparatus for delivering gas and an installation for supplying therapeutic gas, i.e. pure gas or a gas mixture, to a patient, comprising such a gas delivery apparatus that makes it possible to limit the gas consumption, that is, that operates in a similar way to a demand valve, while ensuring minimal inspiratory effort on the part of the patient in order to ensure their respiratory comfort, including during long procedures (e.g. one to two hours or more), and limiting as far as possible the dilution of the therapeutic gas in the event of leaks on the mask, that is, undesirable ingress of ambient air due to defective sealing.

### SUMMARY

One solution according to the invention relates to an apparatus for delivering gas, comprising:
an internal gas passage in fluid communication with a deformable reservoir in order to supply said deformable reservoir with therapeutic gas,
a valve device arranged on the internal gas passage, upstream of the deformable reservoir, in order to control the flow of therapeutic gas circulating in the internal gas passage, and
a control unit with microprocessor controlling the valve device in order to set or adjust the gas flow passing through said valve device and supplying the deformable reservoir with therapeutic gas, and characterized in that it further comprises a pressure sensor configured to:
   perform one or more gas pressure measurements (Pₘₐₛₖ) in a respiratory mask and
   supply the control unit with said gas pressure measurement(s) (Pₘₐₛₖ), and in which the control unit is configured to:
   compare the gas pressure measurement(s) (Pₘₐₛₖ) supplied by the pressure sensor to a given pressure threshold value (P_{threshold}) and
   control the valve device in order to adjust the gas flow as a function of said comparison.

In the context of the invention:
the term “pressure” is used to generally denote a positive pressure (>0 bar), zero pressure (=0 bar) or negative pressure (<0 bar), that is, a vacuum.
pressures are expressed in bar or mbar relative.
the “−” sign before a pressure value denotes that the pressure is negative, that is, that it is a vacuum (i.e. below atmospheric pressure).
the “+” sign before a pressure value denotes that the pressure is positive (i.e. above atmospheric pressure).
the term “therapeutic gas” denotes a gas with one or more gaseous ingredients or compounds, that is, a “pure” gas or a gas mixture.
in “control unit”, the term “unit” is equivalent to the terms “devices”, “apparatus”, “means”, “system” or similar, and the term “control” is equivalent to the terms “command”, “guidance”, “processing” or similar.

Depending on the embodiment considered, the therapeutic gas delivery apparatus of the invention can comprise one or more of the following features:
when the control unit determines that the gas pressure (Pₘₐₛₖ) measured in the mask is less than or equal to the given pressure threshold value (P_{threshold}), i.e. Pₘₐₛₖ≤P_{threshold}, said control unit is configured to control the valve device in order to increase the flow of therapeutic gas passing through said valve device and supplying the deformable reservoir.
the pressure threshold value (P_{threshold}) is less than or equal to 0 mbar.
the pressure threshold value (P_{threshold}) is less than or equal to −0.25 mbar, preferably less than or equal to −0.5 mbar.
the pressure threshold value (P_{threshold}) is stored in the control unit.
the pressure threshold value (P_{threshold}) is stored by the microprocessor or by a data storage memory.
the pressure threshold value (P_{threshold}) is adjustable.
the internal gas passage comprises one or more ducts, pipes or similar.
the pressure sensor comprises a differential pressure sensor.
the pressure sensor is electrically connected to the control unit.
the valve device comprises a proportional valve.
the pressure sensor is configured to supply the control unit with one or more gas pressure measurements (Pₘₐₛₖ), preferably several successive pressure measurements, in the form of numerical values or signals representing such numerical values (for example, voltage signals) which values or signals can be processed as they are or converted into numerical values by the control unit.
the pressure sensor is pneumatically connected, via a pneumatic connection, such as a flexible duct, to a respiratory mask in order to take pressure measurements in the internal respiratory chamber of the mask body.
it comprises a power source of the cord and mains plug type (e.g. 110/220 V) and or an internal battery, preferably rechargeable.
the power source supplies electrical current to the control unit and all of the other components of the apparatus present (depending on the embodiment selected) that require electrical power to operate, for example one or more components such as a display screen, LED, audible and/or visible alarm device, etc.
it comprises a rigid external casing, for example made from a polymer or other material.
the control unit, at least part of the internal gas passage, the deformable reservoir, the pressure sensor and/or the valve device are arranged in the casing.
the deformable reservoir comprises a flexible balloon or similar.
the deformable reservoir deforms as a function of the quantity and/or pressure of therapeutic gas that it contains. It can therefore adopt different states, stages or levels of filling, in particular a so-called “full” stage, a so-called “empty” stage (i.e. minimal residual quantity of gas) and intermediate stages corresponding to partial filling of the reservoir (i.e. between the “full” and “empty” stages).
the pressure sensor is configured or controlled in order to perform pressure measurements (Pₘₐₛₖ) at given time intervals, preferably every 20 msec or less, preferably every 10 msec or less, or even every 5 msec or less.
the control unit is configured to control the valve device, in particular the proportional valve, in order to adjust (i.e. set or modify) the gas flow passing through said valve device as a function of the comparison made by the control unit between the pressure measured at the mask (Pₘₐₛₖ) and the predetermined given pressure threshold value (P_{threshold}) acting as a reference pressure, in particular in order to increase the therapeutic gas flow passing through the valve device and supplying the deformable reservoir, when the control unit determines that the gas pressure (Pₘₐₛₖ) measured in the mask is less than or equal to the given pressure threshold value (P_{threshold}), i.e. Pₘₐₛₖ≤P_{threshold}, where P_{threshold}≤0 mbar, preferably P_{threshold}≤−0.25 mbar, so as to accelerate the filling of said deformable reservoir.
the deformable reservoir is made from a flexible material of the rubber or silicone type or similar, for example a NuSil LSR silicone rubber.
the control unit with microprocessor comprises one or more microprocessors, preferably one (or more) microcontroller(s).
the one (or more) microprocessor(s) use(s) one or more algorithms.
the control unit comprises one or more data storage memories or similar, for example reference tables.
the control unit with microprocessor comprises an electronic board holding the one or more microprocessors, preferably one or more microcontrollers.
the gas delivery apparatus further comprises a flow meter or flow sensor arranged in the internal gas passage in order to measure the gas flow circulating in said internal gas passage.
the flow sensor (i.e. flow meter) is arranged in the internal gas passage, downstream of the valve device, in particular the proportional valve, so that it can measure the gas flow supplied by said valve device.
the flow sensor is arranged upstream of the deformable reservoir, preferably upstream of the connection point of the air inlet line.
the flow sensor is electrically connected to the control unit and supplies it with the measurements that it performs.
the flow sensor is a mass-flow sensor or a differential pressure sensor.
it comprises a non-return device arranged in the internal air passage, downstream of the reservoir, preferably a non-return valve.
the deformable reservoir has a volume of between 0.1 and 3 L, measured at rest (i.e. internal pressure equal to atmospheric pressure).
the deformable reservoir has a wall with a thickness of between 0.10 and 0.90 mm, typically between 0.25 and 0.75 mm.
the apparatus further comprises one (or more) one-way valve(s) arranged in the internal gas passage, in particular downstream of the deformable reservoir.
the apparatus further comprises a human-machine interface (HMI) comprising an information display screen, preferably a touch screen, and/or one or more selection keys or buttons, particularly virtual keys that are displayed on the touch screen, and/or a starting device, such as an on/off button, and/or other elements.
the apparatus further comprises an alarm system for alerting the user in the event of a problem affecting the apparatus or the gas, for example a valve or sensor fault, an incorrect gas composition (e.g. hypoxic mixture) or other problem. The alarm system can comprise means or a device for emitting audible and/or visible signals.

The installation for delivering therapeutic gas to a patient comprises a gas delivery apparatus according to the invention and a respiratory mask, said respiratory mask being in fluid communication with the deformable reservoir and supplied with therapeutic gas by said deformable reservoir, and also pneumatically connected to the pressure sensor in order to make it possible for pressure measurements to be taken in the mask

Depending on the embodiment considered, the therapeutic gas supply installation according to the invention can comprise one or more of the following features:
the respiratory mask is a face mask (i.e. naso-buccal) covering the patient's nose and mouth, in use, that is, when it is worn by said patient.
it further comprises a therapeutic gas source fluidly connected to the internal gas passage in order to supply said gas passage with therapeutic gas.
the therapeutic gas source comprises one or more gas containers, particularly cylinders.
the therapeutic gas source comprises a gas container containing an O₂/N₂O gas mixture, preferably an equimolar O₂/N₂O mixture (i.e. 50 mol %/50 mol %).
alternatively, the therapeutic gas source comprises a gas container containing an O₂/argon gas mixture, preferably containing 35 to 45 vol % 02 and 55 to 65 vol % Ar, for example a mixture containing 38 to 43 vol % 02 and 57 to 62 vol % Ar, particularly a binary mixture made up of 40 vol % 02 and 60 vol % Ar.
alternatively, the therapeutic gas source comprises a first gas container containing argon or N₂O, a second gas container containing oxygen (O₂) and a gas mixer supplied with gas by said first and second gas containers, said mixer performing the mixing of the gases coming from the first and second gas containers in order to obtain an O₂/N₂O or O₂/argon gas mixture.
it comprises a pressure supply duct, i.e. a pneumatic connection, arranged between the respiratory mask and the pressure sensor of the gas delivery apparatus.

### BRIEF DESCRIPTION OF THE DRAWINGS

The invention will now be better understood from the following detailed description, which is given by way of a non-limiting illustration, with reference to the appended figures, in which:

FIG. 1 schematically shows one embodiment of a gas supply installation according to the invention,

FIG. 2 schematically shows one embodiment of the internal architecture of a gas delivery apparatus according to the invention,

FIG. 3 illustrates the operation of the control unit of the gas delivery apparatus in FIG. 2, in particular the pressure and flow curves obtained over time, and

FIG. 4 is a comparison of the performance of a gas delivery apparatus according to the present invention and several other devices.

### DETAILED DESCRIPTION OF PREFERRED EMBODIMENTS

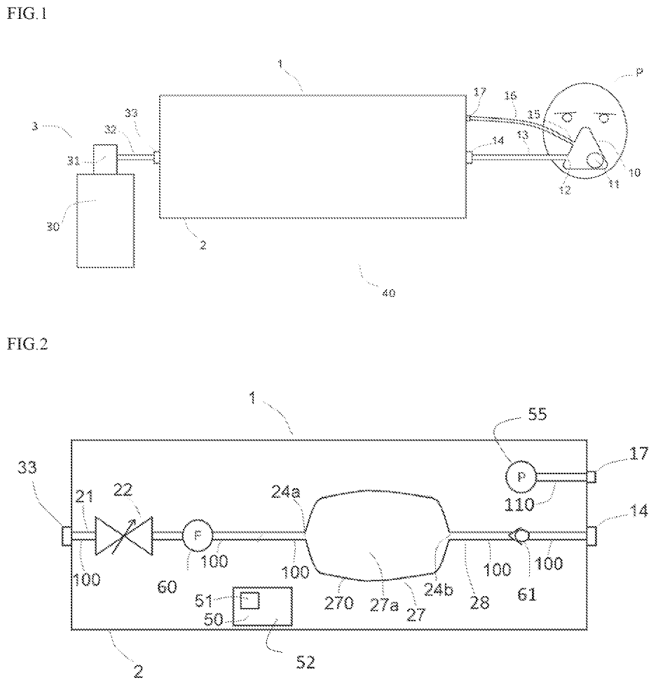

FIG. 1 schematically shows one embodiment of a gas supply installation 1 according to the present invention. It comprises a gas delivery apparatus 1 according to the invention, in particular as schematically shown in FIG. 2, comprising an external casing 2 forming a rigid shell, for example made from a polymer, comprising the internal components, particularly an internal gas passage, a deformable reservoir, a valve device and a control unit with microprocessor as explained below.

A therapeutic gas source 3, such as a gas cylinder 30 provided with a valve 31, supplies a therapeutic gas, that is, a gas or gas mixture, to the gas delivery apparatus 1 via a connecting hose 32, connected to the inlet port 33 of the gas delivery apparatus 1.

The therapeutic gas passes through the gas delivery apparatus 1, as explained below, in order to then be delivered to a patient P by means of a gas pipe 13 that is fluidly connected to an outlet port 14 of the gas delivery apparatus 1. The gas is supplied to the patient P via a respiratory interface or mask 10 supplied by the gas pipe 13, such as a flexible tube.

Preferably, the respiratory interface or mask 10 is a face mask, i.e. a naso-buccal mask, covering the patient's mouth and nose. Other respiratory interfaces could of course be suitable.

The face mask 10 has an exhalation port 11 and an inhalation port 12. The inhalation port 12 is fluidly connected to the gas pipe 13 that conveys the gas. The exhalation port 11 preferably comprises a non-return valve that directs and makes it possible to discharge the gases to the atmosphere when the patient exhales, that is, CO₂-rich exhaled gases, and also prevents ambient air from entering the mask 10 when the patient inhales the therapeutic gas, that is, during their inspiratory phases. The non-return valve comprises a one-way valve, such as a silicone disc resting on a perforated surface, which only allows the gas to pass through in one direction, for example the one-way valve with part reference 97351 sold by Qosina.

The mask 10 further has a pressure tapping port 15 fluidly connected to a pressure supply line or duct 16, such as flexible tube, for example a silicone tube several metres long, in turn pneumatically connected to the pressure sensor 55, via a measuring port 17 arranged on the casing 2 of the gas delivery apparatus 1. This configuration allows the pressure sensor 55 arranged in the casing 2 to take pressure measurements in the mask 10 in order to monitor the pressure (i.e. vacuum) prevailing therein, as explained below.

The gas source 3 contains a pressurized therapeutic gas, for example an argon/oxygen mixture, for example comprising 60 vol % argon and 40 vol % oxygen, at a maximum pressure of the order of 250 bar. The valve 31 is preferably an integrated pressure regulator valve delivering the gas to the connecting hose 32 at a reduced pressure, for example of the order of 5 bar. The integrated pressure regulator 31 is preferably protected by a rigid cap (not shown).

FIG. 2 schematically shows one embodiment of the internal architecture of the gas delivery apparatus 1 according to the present invention that forms part of the gas supply installation 40 according to the present invention, which is schematically shown in FIG. 1.

The gas delivery apparatus 1 comprises a control unit 50 comprising a microprocessor 51 held by an electronic board 52 used to control a valve device 22, such as a proportional valve, in order to set or adjust the gas flow passing through said valve device 22, as explained below.

The control unit 50 comprises one (or more) microprocessor(s) 51, typically one (or more) microcontroller(s), executing one (or more) algorithm(s) that receive(s) and analyse(s) the measurements supplied by various sensors, in particular by the pressure sensor 55 arranged in the casing 2 and pneumatically connected to the mask 10 by the pressure supply line 16.

An internal gas passage 100, for example a duct or similar, is arranged in the casing 2 and extends between an inlet port or orifice 33 and an outlet port or orifice 14 so as to convey the therapeutic gas from the inlet port 33 to the outlet port 14 and then allow it to be conveyed to the mask 10, via the flexible pipe 13.

The valve device 22, namely here a proportional valve, is arranged in the internal gas passage 100, preferably in the upstream section 21 of said internal gas passage 100. It is controlled by the microcontroller 51 of the control unit 50 in order to modify the therapeutic gas flow passing through said valve device 22 and circulating in the lumen of the internal gas passage 100 towards the outlet port or orifice 14, as described below.

Various types of proportional valve can be used as a valve device 22; preferably, a proportional valve that operates over a wide flow range is selected, for example the valve referred to as IMI FAS FLATPROP.

A flow sensor 60 is arranged in the internal gas passage 100, at the outlet of the valve device 22, in order to measure the therapeutic gas flow delivered by said valve device 22, typically a proportional valve. The flow sensor 60 can be a mass-flow sensor or based on a differential pressure sensor.

The flow sensor 60 is electrically connected to the control unit 50 and delivers a flow signal that is processed by said control unit 50, typically by the microprocessor 51, preferably a microcontroller.

Preferably, a volumetric flow rate is obtained after conversion of the signal supplied by the flow sensor 60 using a specific look-up table stored in a memory interacting with the control unit 50. The flow sensor 60 can also be used to detect any faults on the proportional valve 22 or to determine the quantity of gas, that is the volume, delivered by the gas source 3.

The internal gas passage 100 then conveys the gas to a deformable reservoir 27, in particular a flexible reservoir, positioned downstream of the flow sensor 60, and fluidly connected to said gas passage 100.

The deformable reservoir 27 comprises a flexible peripheral wall 270 defining an internal volume 27a for the gas, forming a deformable pouch for the therapeutic gas. At rest, the internal volume 27a is for example between approximately 0.2 and 1 L.

The gas flow enters the internal volume 27a of the deformable reservoir 27 through a reservoir inlet orifice 24a, in fluid communication with the internal gas passage 100. Preferably, the properties of the deformable reservoir 27 are such that it is highly deformable. For example, its peripheral wall 270 has a thickness of between approximately 0.25 and 0.75 mm and is made from a biocompatible flexible silicone, for example a silicone from the LSR range sold by NuSil.

The gas leaves the reservoir 27 through a reservoir outlet orifice 24b that is fluidly connected to a downstream section 28 of the internal gas passage 100, extending to the outlet port 14.

One (or more) non-return device(s) 61, such as a non-return valve, is positioned in the internal gas passage 100, downstream of the reservoir 27, namely between the outlet orifice 24b of the reservoir 27 and the outlet port 14 of the casing 2, in order to prevent any backflow of gas. The gases exhaled by the patient P are thus discharged solely through the exhalation port 11 of the mask 10 and cannot return to the reservoir 27.

The non-return valve 61 is preferably designed so that a very small drop in pressure, typically less than or equal to 0.2 mbar, is generated through it, when a gas flow passes through it.

Of course, several non-return valves 61 can be used instead of just one, for example 3 to 5 positioned in parallel (not shown).

In order to make it possible to take pressure measurements in the mask 10, a pressure sensor 55, preferably a differential pressure sensor, is provided in the casing 2 of the apparatus 1. The pressure sensor 55 is configured to measure negative pressures (that is, pressures below atmospheric pressure or vacuums) down to approximately −5 mb.

Here, the pressure sensor 55 is a differential pressure sensor that comprises two detection orifices comprising a first detection orifice kept in atmospheric conditions (that is, at atmospheric pressure, i.e. 1 atm) and a second detection orifice positioned in a measuring duct 110, connected to the measuring port 17 of the casing 2. The measuring port 17 is fluidly connected to the pressure supply line 16 connected to the mask 10 in order to monitor the pressure prevailing in the respiratory chamber of said mask 10. For example, the differential pressure sensor with part reference SPD3X available from Sensirion can be used.

At successive time intervals, for example at a frequency of 5 msec, the differential pressure sensor 55 sends a pressure measurement signal Pₘₐₛₖ to the control unit 50, which signal Pₘₐₛₖ reflects the pressure measured in the mask 10 at the time in question. The control unit 50 then processes this pressure signal in order to control the proportional valve 22 as set out in detail below, in order to adjust the gas flow sent to the flexible reservoir 27.

The flexible reservoir 27 has various inflation/deflation states as a function of the gas pressure prevailing therein, and therefore as a function of the quantity of gas that is introduced into it or withdrawn from it, comprising at least:
a so-called “rest” state, in which the internal volume 27a, filled with gas, is at atmospheric pressure (i.e. 1 atm).
a so-called “inflated” state, in which the internal volume 27a, filled with gas, is at a pressure higher than ambient pressure (that is, >1 atm).
“partially deflated” states, in which some of the gas contained in the reservoir has left it.

A power source (not shown) supplies electrical current to all of the components that operate using electrical energy, such as sensors, control unit, controlled valves, human-machine interface (HMI), digital display screen, etc. It can be positioned in the casing 2, for example a rechargeable battery, or comprises a cord and a mains plug (110/220 V), and optionally a current converter.

During therapy with administration of therapeutic gas, the patient P performs a succession of inspirations and exhalations in order to inhale the therapeutic gas, for example an O₂/argon or N₂O/O₂ mixture, and exhale the CO₂-rich gases resulting from the pulmonary exchanges.

In order to facilitate understanding of how the apparatus 1 operates, it is considered that:
the pressure in the internal volume 27a of the reservoir 27 is equal to atmospheric pressure, that is, the reservoir 27 is in the rest position.
the reservoir 27 is filled with the therapeutic gas coming from the gas source 3.
the patient is initiating an inspiration.

When the patient starts to inspire, the exhalation port 11 of the mask 10 is closed and a slight vacuum occurs at the inhalation port 12 of the mask. This vacuum spreads to the differential pressure sensor 55, respectively via the pressure supply line 16, the measuring port 17 and the measuring duct 110. The pressure information is then transmitted by the differential pressure sensor 55 to the processing unit 50, in particular to the microprocessor 51.

In addition, this vacuum spreads in parallel in the pipe 13, the outlet port 14 and the downstream section 28 of the internal gas passage 100.

When the gas pressure in the internal volume 27a of the reservoir 27 is equal to atmospheric pressure (i.e. 1 atm), a positive differential pressure then occurs through the non-return valve 61, which allows a certain quantity of gas to pass through said non-return valve 61, in order to meet the patient's respiratory demand.

In other words, a flow of gas can be established from the reservoir 27 towards the mask 10. As a result, the internal volume 27a of the reservoir 27 then empties and the reservoir 27 deflates, in turn creating a slight vacuum in the internal volume 27a.

The control unit 50 is configured to ensure that at any time, the pressure prevailing in the mask 10 is as close as possible to atmospheric pressure (i.e. 1 atm), i.e. 0 mbar relative. To do this, the control unit 50 controls the proportional valve 22 so that the flow supplied by said proportional valve 22 is proportional to the pressure Pₘₐₛₖ measured in the mask 10 by the differential pressure sensor 55.

To this end, the microprocessor 51 can for example implement an algorithm of the following type:
If “Mask pressure” is negative: Flow (L/min)=α*|P|
If “Mask pressure” is positive: Flow (L/min)=0
where: α is a positive constant and |P| is the absolute value of the pressure measured in the mask.

The control unit 50 therefore only acts on the proportional valve 22 if the pressure prevailing in the mask 10 is negative, that is, the proportional valve 22 is controlled to or stays in the closed position as soon as the pressure in the mask 10 becomes positive.

If the case of negative pressure (i.e. vacuum) in the mask 10, measured by the differential pressure sensor 55, reflecting inspiration by the patient P, the proportional valve 22 will be controlled by the control unit 50, in particular by the microprocessor 51, such that: Flow (L/min)=α*|P|.

A proportionality then occurs between the flow delivered by the proportional valve 22 and the negative pressure measured in the mask 10 by the differential pressure sensor 55. The further away the pressure value moves from 0 mb, the higher the flow. Conversely, the closer the pressure value moves to 0 mb, the lower the flow. It will of course be understood that the algorithm described here is for illustration, and that more sophisticated algorithms such as control by proportional, integral and derivative terms (PID) could be implemented.

Furthermore, the gas delivery apparatus 1 can comprise other elements, such as a human-machine interface (HMI) with information display screen, preferably a touch screen, one or more selection keys or buttons, a starting device, such as an on/off button, an alarm system and/or other elements.

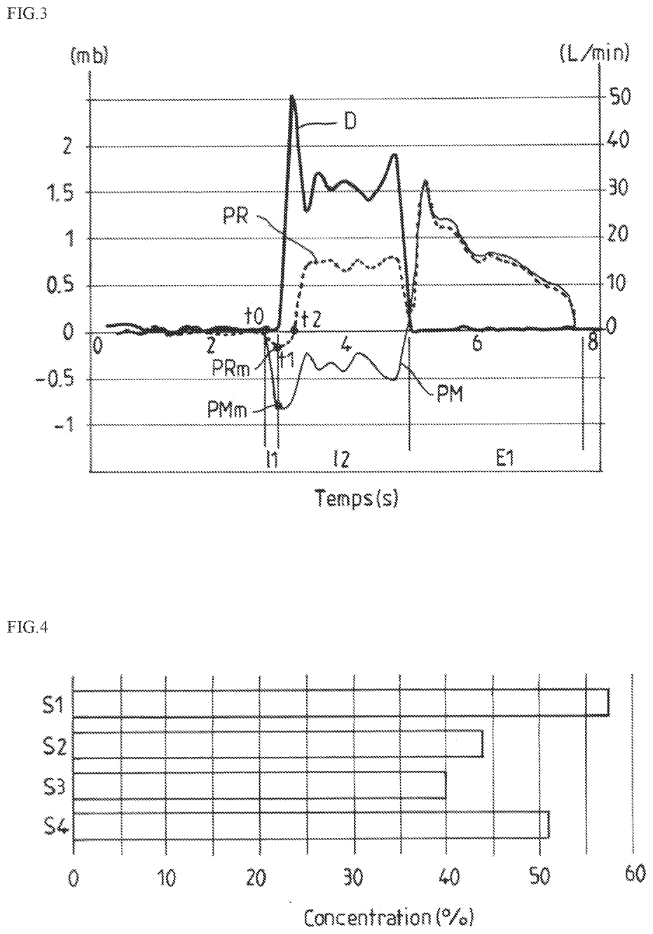

FIG. 3 schematically shows the operation of the control unit 50, in particular of the algorithm implemented by the microprocessor 51 of the gas delivery apparatus 1, in response to an inspiration by the patient P.

The inspiration by the patient P is split into two successive distinct portions I1 and I2, where I1 corresponds to the very start of the inspiration. If t0 is the exact time of the start of the inspiration by the patient P, at this time the relative pressure PR in the reservoir 27 is thus zero, that is, atmospheric pressure (i.e. 1 atm).

As mentioned above, the inspiration by the patient then creates a vacuum in the mask 10, which is represented by the curve PM. In response to this vacuum, the control unit 50 will control the proportional valve 22 to adjust the therapeutic gas flow in order to limit the pressure drop in the mask 10.

As in any system incorporating electromechanical elements, there is an intrinsic response time, that it a delay, in response to the physical manifestation, which here is the vacuum in the mask 10.

During this phase I1, the pressure in the deformable reservoir 27 decreases, which is a sign that it is deflating and that a quantity of gas is circulating through the non-return valve 61 towards the mask 10, in order to meet the inspiratory demand of the patient P. At t1, this pressure decrease in the reservoir 27 reaches a minimum value PRm and, similarly, a minimum pressure PMm occurs in the mask 10.

This time t1 corresponds to the moment when the proportional solenoid valve 22 starts to open in response to the demand by the control unit 50 and therefore to deliver a flow D, marking the transition to phase I2.

In these conditions, the gas flow D will meet the need of the patient P and at the same time fill the reservoir 27, the pressure PR of which will increase until it returns to zero at t2, which is a sign that the reservoir 27 has returned to its rest state, that is, completely filled.

This increase in pressure PR in the reservoir 27 is naturally accompanied, at the same time, by an increase in the pressure PM in the mask 10, here close to −0.5 mb.

The portion of phase I2 subsequent to t2 sees the reservoir 27 return to an over-inflated situation as the pressure PR is positive, which is perfectly normal.

The control unit 50 controls the proportional valve 22 to adjust the flow passing through it so that the pressure PM in the mask 10 is as close to 0 as possible. The downstream elements of the reservoir 27, particularly the downstream section 28 of the passage 100, the non-return valve 61 and the pipe 13, create resistance to the flow of the gas. In relation to the inspiratory demand of the patient P (that is, their inspiratory flow), this flow resistance is equal to the difference between the pressure PR in the reservoir 27 and the pressure PM in the mask 10.

In other words, a positive pressure in the reservoir 27 has the sole aim of compensating for all or part of the flow resistance of the aforementioned elements so that the negative pressure in the mask 10 is as close as possible to 0.

If the apparatus 1 did not have a flow delivery mechanism based on the pressure prevailing in the mask 10, that is, if the reservoir 27 emptied progressively in response to the inspiration by the patient P, then the vacuum in the mask 10 allowing the patient P to meet their respiratory needs would be equal to the sum of the flow resistances of the elements situated downstream of the reservoir 27, including the reservoir 27 itself.

Finally, phase I2 gives way to an expiratory phase E1, in which the patient exhales through the exhalation port 11 of the mask 10. This exhalation then generates a positive pressure PM in the mask 10 and the control unit 50 then controls the proportional valve 22 so as to interrupt the delivery of gas, that is, the flow. At the same time, the reservoir 27, itself at positive pressure PR, empties progressively following the profile of the pressure PM prevailing in the mask 10.

The reservoir 27 is essential to the satisfactory operation of the apparatus 1. If it was not present, the gas would circulate in rigid, that is non-deformable, elements, such as the internal gas passage 100 and the pipe 13. During phase I1, before the proportional valve 22 opens, the patient's respiratory demand would thus not be satisfied, resulting in major respiratory discomfort for the patient. In addition, throughout the inspiratory phase, the reservoir 27 acts as a buffer by attenuating the effect of the variations in ventilatory demand of the patient P and of the response of the control unit 50 and the proportional valve 22 to these variations.

FIG. 4 compares the performance of a gas delivery apparatus 1 according to the present invention and several prior art devices, such as a continuous flow system and a demand valve.

In order to obtain representative data, this comparison implements a test bench comprising an “electronic patient”, namely a device that mimics the respiration of a patient, for example the ASL 5000 breathing simulator available from Ingmar Medical, which makes it possible to repeatably simulate the respiration of a patient.

The different devices tested are connected to the “electronic patient” by means of a gas conveying duct with a calibrated orifice simulating a leak in the respiratory mask.

The therapeutic gas source supplies a mixture made up of 60% argon and 40% oxygen (vol %).

As a function of the resistance of each of the devices, and therefore of the vacuum generated by the “electronic patient”, it is possible to measure the therapeutic gas concentration inhaled by the patient under the effect of the dilution with the ambient air.

In FIG. 4:
S1 is the gas delivery apparatus 1 of the present invention, shown schematically in FIG. 1 and FIG. 2;
S2 is a demand valve, for example the GCE Ease II demand valve;
S3 is a continuous flow system, for example the system available from Intersurgical; and
S4 is a gas delivery apparatus similar to the apparatus of the invention (i.e. S1) but in which the system for limiting the vacuum in the mask has been eliminated (i.e. sensor, pressure supply line, etc.).

The results obtained clearly show the limitations of the current systems.

Under the effect of the simulated leak, the argon concentration inhaled by the patient P is thus close to 40% (vol %) for the continuous flow system S3 and 45% for the demand valve S2, namely a loss of 20% and 15% of argon volume respectively, which does not make it possible to ensure the efficacy of the device during delivery of gas to a patient as the argon content supplied to the patient is far below that expected, i.e. 60 vol %.

Conversely, the gas delivery apparatus 1 (S1) of the invention makes it possible to greatly limit the dilution with the ambient air, maintaining, in the same test conditions, a concentration of the order of 57 vol %, namely approximately the desired content (i.e. 60%), thus fully ensuring therapeutic efficacy.

By way of comparison, the gas delivery apparatus 1 stripped of the means for limiting the vacuum in the mask (S4) remains superior to the existing devices (S2, S3) but only ensures a concentration slightly greater than 50 vol %, which is insufficient to ensure efficacy of the argon treatment, for which an effective content of 60 vol % is desired.

The gas delivery apparatus 1 according to the invention therefore meets the needs of patient comfort and minimizing the impact of leaks in terms of the reduction in the concentration of the inhaled gases in every respect, thus ensuring the desired therapeutic efficacy.

This level of efficacy is only possible by combining a deformable reservoir 27 with control of the gas flow delivered as a function of the pressure measured in the mask 10 by the pressure sensor 55 that interacts with the control unit 50 with microprocessor 51.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1. A gas delivery apparatus (1) comprising:
an internal gas passage (100) in fluid communication with a deformable reservoir (27) configured to be able to supply the deformable reservoir (27) with a therapeutic gas,
a valve device (22) arranged on the internal gas passage (100), upstream of the deformable reservoir (27), adapted for and configured to control a flow of therapeutic gas circulating in the internal gas passage (100), and
a control unit (50) with a microprocessor (51) adapted for and configured to control the valve device (22) to set or adjust the therapeutic gas flow passing through said valve device (22), thereby supplying the deformable reservoir (27) with the therapeutic gas,
wherein the gas delivery apparatus (1) further comprises a pressure sensor (55) adapted for and configured to:
a) perform one or more gas pressure measurements (Pₘₐₛₖ) in a respiratory mask (10) and
b) supply the control unit (50) with said gas pressure measurement(s) (Pₘₐₛₖ),
and in which the control unit (50) is adapted for and configured to:
I) compare the gas pressure measurement(s) (Pₘₐₛₖ) supplied by the pressure sensor (55) to a given pressure threshold value (P_{threshold})
II) and control the valve device (22) to adjust the therapeutic gas flow as a function of said comparison.

2. The gas delivery apparatus (1) according to claim 1, wherein when the control unit (50) determines that the gas pressure (Pₘₐₛₖ) measured in the respiratory mask (10) is less than or equal to the given pressure threshold value (P_{threshold}), (Pₘₐₛₖ≤P_{threshold}), said control unit (50) is adapted for and configured to control the valve device (22) to increase the flow of therapeutic gas passing through said valve device (22) and supplying the deformable reservoir (27).

3. The gas delivery apparatus (1) according to claim 2, wherein the pressure threshold value (P_{threshold}) is less than or equal to 0 mbar.

4. The gas delivery apparatus (1) according to claim 3, wherein the pressure threshold value (P_{threshold}) is less than or equal to −0.25 mbar.

5. The gas delivery apparatus (1) according to claim 1, wherein the pressure threshold value (P_{threshold}) is stored in the control unit (50) by the microprocessor (51) of the control unit (50).

6. The gas delivery apparatus (1) according to claim 1, characterized in that the pressure sensor (55) comprises a differential pressure sensor.

7. The gas delivery apparatus (1) according to claim 1, wherein the valve device (22) comprises a proportional valve.

8. The gas delivery apparatus (1) according to claim 1, further comprising a flow sensor (60) arranged in the internal gas passage (100) downstream of the valve device (22) and adapted for and configures to measure the therapeutic gas flow circulating in said internal gas passage (100).

9. The gas delivery apparatus (1) according to claim 1, further comprising a non-return device (61) arranged in the internal gas passage (100), downstream of the deformable reservoir (27).

10. An installation for supplying therapeutic gas (40) to a patient comprising a gas delivery apparatus (1) according claim 1 and a respiratory mask (10), said respiratory mask (10) being in fluid communication with the deformable reservoir (27) and supplied with the therapeutic gas by said deformable reservoir (27), said respiratory mask (10) pneumatically connected to the pressure sensor (55).
